(19) United States
(12) Patent Application Publication
(10) Pub. No.: US 2025/0387140 A1
(43) Pub. Date: Dec. 25, 2025

(54) CERVICAL SEAL FOR TRANSCERVICAL ENDOSCOPY

(71) Applicant: Csaba TRUCKAI, San Jose, CA (US)

(72) Inventor: Csaba TRUCKAI, San Jose, CA (US)

(21) Appl. No.: 19/243,484

(22) Filed: Jun. 19, 2025

Related U.S. Application Data
(60) Provisional application No. 63/661,644, filed on Jun. 19, 2024.

(51) Int. Cl. A61B 17/42; A61B 17/00

(52) U.S. Cl. CPC A61B 17/42; A61B 17/00234; A61B 2017/00296; A61B 2017/4225

(57) ABSTRACT

Cervical seals for use with an endoscope or other tools configured for transcervical access to a patient's uterine cavity, including c cervical seal for use during transcervical access procedures with an elongate device adapted to fit over a shaft of an endoscope; a sealing member disposed at a distal end of the elongate device and an elongate helical spring disposed within the elongate device.

### CROSS-REFERENCE TO RELATED APPLICATIONS

This application is a non-provisional of U.S. Provisional application No. 63/661,644 filed Jun. 19, 2024, the entirety of which is incorporated by reference.

### FIELD OF THE INVENTION

This disclosure relates generally to the field of gynecology and, more specifically, to a cervical seal for use with an endoscope or other tools configured for transcervical access to a patient's uterine cavity.

### BACKGROUND OF THE INVENTION

Minimally invasive techniques in gynecology are well known and involve accessing the uterine cavity through the cervix, eliminating the need for traditional laparoscopic or abdominal incisions. In such a procedure, an endoscope is introduced through the cervix to provide for viewing the uterine cavity. A distention fluid such as saline is introduced through the endoscope to distend the uterine cavity. Various cervical sealing devices have been invented to provide a seal in the cervix around the endoscope to prevent leakage of distension fluid.

### SUMMARY

The present disclosure includes methods and devices for providing a seal against tissue within a cavity that has an opening. For example, such a sealing device can comprise a seal against a cervix.

In one example, the present disclosure includes a cervical seal device for use with an endoscope during transcervical access procedures. For example, such a device can include an elongate body adapted to fit over a shaft of an endoscope; a sealing member disposed at a distal end of the elongate body, the sealing member having a distal surface with a tapered configuration that tapers in a distal direction; and the elongate body having a helical spring configuration such that the elongate body can compress when a force is applied and expand upon reduction of the force, a proximal end of the elongate body adapted to abut against a handle of the endoscope and a distal end of the helical spring configuration connected to the sealing member, wherein the elongate body is configured to bias the sealing member toward a cervix of a patient to maintain sealing engagement with the cervix.

A variation of the cervical seal includes a sealing member that comprises a housing carrying an elastomeric seal configured to create a seal against the shaft extending through an aperture in the sealing member.

In an additional variation, the sealing member comprises an internal housing located within an external housing, where the external housing comprises the tapered configuration and is configured to maintain a sealing engagement with the cervix. Variations of the device include the internal housing and external housing being configured to rotate relative to each other about a longitudinal axis such that when engaged against the cervix, the external housing remains stationary upon rotation of the internal housing.

The devices can further include a collar located at the proximal end of the elongate body, the collar configured to abut a portion of a handle of the endoscope. The collar can optionally include a locking mechanism having a locking surface that can be removably locked against the shaft. The locking mechanism can permit locking of the collar on the shaft after shortening of an axial distance between the proximal end of the elongate body and the sealing member. The locking mechanism can comprise a rocker structure, where the locking surface is positioned within the collar.

Variations of the devices can include cervical seals wherein the distal surface comprises a soft material to improve conformability of the distal surface against the cervix.

The present disclosure also includes methods of endoscopically examining a cavity within a body of a patient. For example, the methods can include advancing an endoscope within the body, the endoscope having a sealing body positioned over a shaft of the endoscope with a sealing member of the sealing body located at a distal portion of the shaft and a proximal end of the sealing body extending proximally from the sealing member and comprising a spring structure; positioning the endoscope within the body such that the sealing member forms a fluid seal against a tissue surface about an opening within the cavity; and advancing the shaft through the sealing member such that the spring structure compresses while the shaft is moved therethrough and causes the sealing member to apply force against the tissue surface to maintain the fluid seal of the sealing member against the tissue surface.

The methods can optionally include compressing the spring structure in a longitudinal direction and locking a proximal end of the sealing body to a portion of the shaft to increase the force applied by the sealing member against the tissue surface.

### BRIEF DESCRIPTION OF THE DRAWINGS

FIG. 1 illustrates a perspective view of a variation of a cervical seal configured for positioning around an elongated endoscope shaft.

FIG. 2 is a side view of the cervical seal of FIG. 1 disposed around an endoscope shaft.

FIG. 3 is a section view of a distal portion of the cervical seal of FIGS. 1 and 2.

FIG. 4 is a perspective view of a proximal end of another variation of a cervical seal with a locking collar.

FIGS. 5A and 5B illustrate an example of using a cervical seal with an endoscope to perform an endoscopic examination.

### DETAILED DESCRIPTION OF THE INVENTION

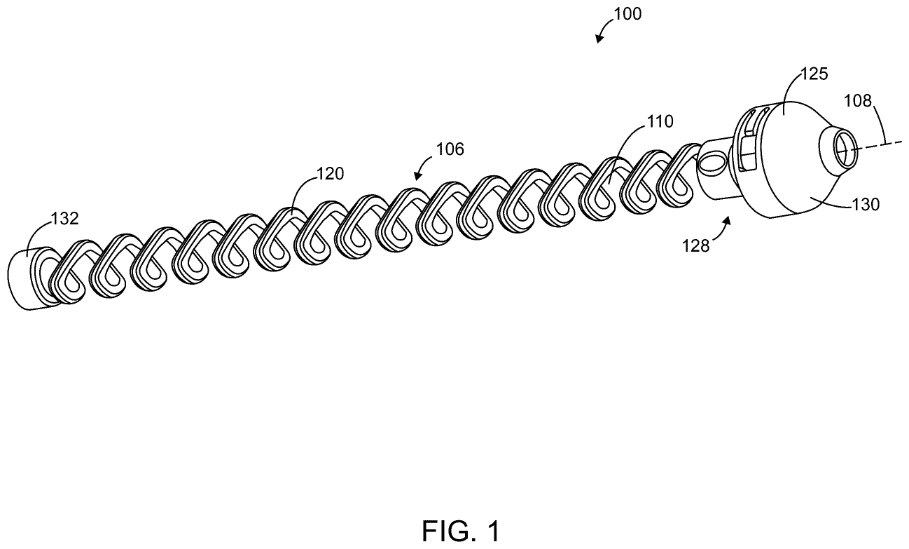

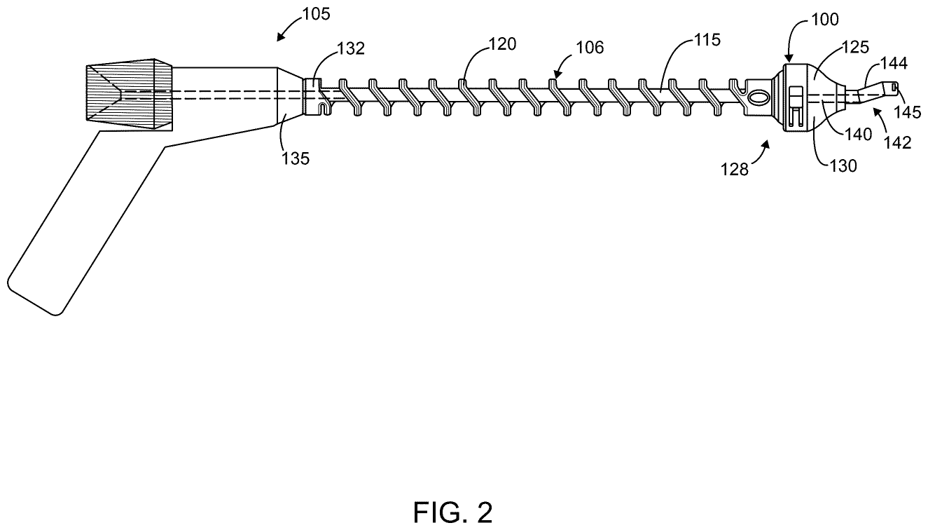

Referring to FIGS. 1 and 2, a cervical seal 100 is shown that is adapted for use with an endoscope 105 (FIG. 2). The cervical seal 100 comprises an elongate body 106 extending about longitudinal axis 108 with a central passageway 110 adapted to and fit (e.g., positioned or coupled) over an elongate shaft 115 of the endoscope 105 by sliding or translation of the body 106 over the endoscope shaft 115. The elongate body 106 of the cervical seal 100 includes a sealing member 125 at a distal end 128 with a helical spring structure or structure 120 extending proximally from the distal portion 128. The helical spring structure 120 can be formed from a biocompatible plastic, a metal, or a combination thereof, that allows compression, upon application of a force, and expansion, upon reduction of the force, of the spring structure 120 of the elongate body 106 to accommodate the movement of the endoscope shaft while maintaining a force of the sealing member 125 against tissue.

As shown in FIGS. 1 and 2, the sealing member 125 is disposed at the distal end 128 of the elongate seal 100. The sealing member 125 has a tapered or angled configuration that tapers in the distal direction. The tapered shape allows for effective engagement with the cervix to create an effective seal. The distal surface 130 of the sealing member 125 may be formed from a soft material to ensure proper conformability to the cervix.

The helical spring structure 120 has a suitable axial length for use with a selected endoscope 105. The proximal end 132 of the helical spring structure 120 is adapted to abut the endoscope handle 135, as shown in FIG. 2. While the proximal end 132 shown includes a collar structure, variations of the cervical seal 100 can include an elongate body 106 that comprises a spring structure 120 terminating in an open or closed coil of the spring structure 120, where the terminating coil abuts against the endoscope handle.

The endoscope 105 illustrated in FIG. 2 can be of the type disclosed in commonly-owned U.S. Pat. Nos. 11,937,787, 11,596,298, 11,096,560, 11,529,048, 11,089,951, 11,369,253, 11,259,695, 10,432,717, 11,589,736, 11,019,987, 11,432,717, 11,717,141, 11,832,786 and 11,304,594 each incorporated by reference. FIG. 2 shows an endoscope 105 with a working channel 140 and a distal end 142 with an “S” curve 144 that is configured to offset the image sensor 145 from the straight working channel 140. In this variation, the cross-section of the central passageway 110 in the seal 100 is large enough to receive the “S” curve 144 of the shaft 115 of endoscope 105.

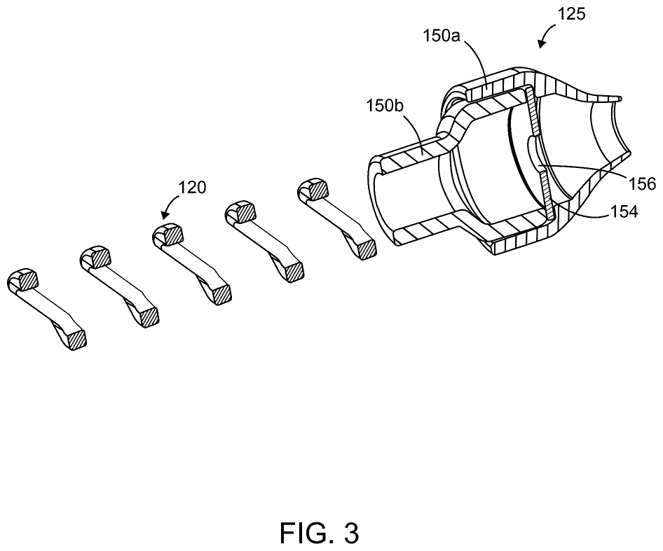

As can be seen in the sectional view of FIG. 3, the sealing member 125 comprises an outer housing 150a and an inner housing 150b that, when assembled, locks an elastomeric shaft seal 154 in the interior of the sealing member 125. The shaft seal 154 has a central opening 156 or a slit for receiving and sealing against the endoscope shaft 115. The outer housing 150a and inner housing 15b can be locked relative to each other, lockable relative to each other, and/or rotatable relative to each other about the longitudinal axis (108 FIG. 1) of the cervical seal 100.

In a method of use, the cervical seal 100 is positioned over the shaft 115 of the endoscope 105, as shown in FIG. 2. It can be understood that the helical spring structure 120 allows for axial movement of the sealing member 125 over the endoscope shaft 115. In use, the physician will manipulate the endoscope shaft 115 during the procedure, and the spring structure 120 will compress and expand to accommodate the movement of the endoscope shaft while maintaining a constant force on the sealing member 125 against the patient's cervix. Thus, the spring urges the sealing member distally to ensure a continuous and reliable seal against a cervix throughout a procedure. In one variation, the ability of the outer housing 150a to rotate relative to the inner housing 150b allows for the outer housing 150a of the sealing member 125 to remain stationary as the endoscope shaft and inner housing 150b are rotated.

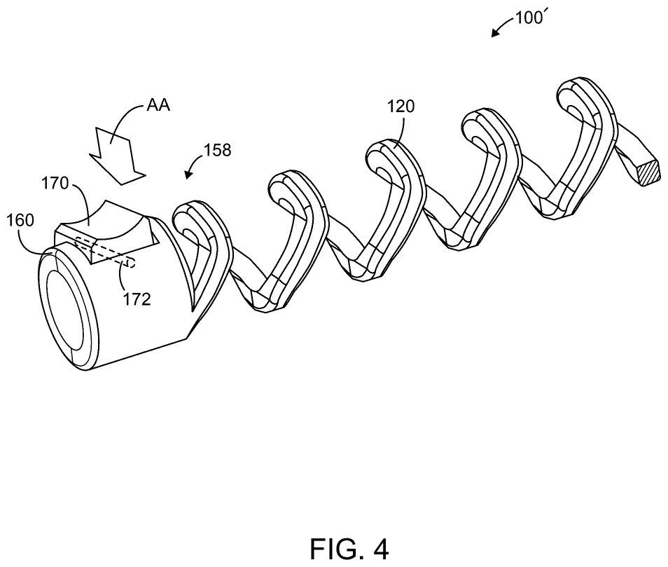

In another variation, referring to FIG. 4, the proximal end 158 of the spring structure 120 comprises a locking collar 160 configured to lock against the shaft 115 of an endoscope 105 of the type shown in FIG. 2. The locking collar 160 allows the physician to shorten the axial distance between the proximal end 158 of the spring structure 120 and a cervix (or the sealing member 125) to thereby apply greater pressure on the sealing member 125 when engaging a cervix. The locking collar can comprise any form a lock mechanism and is shown with a rocker 170 that can be click stopped in an unlocked position shown in FIG. 4 or the rocker 170 can be pressed in the direction of arrow AA in FIG. 4 to be click stopped or ratcheted in a locking position with a locking surface of the rocker portion 170 pressed against an endoscope shaft.

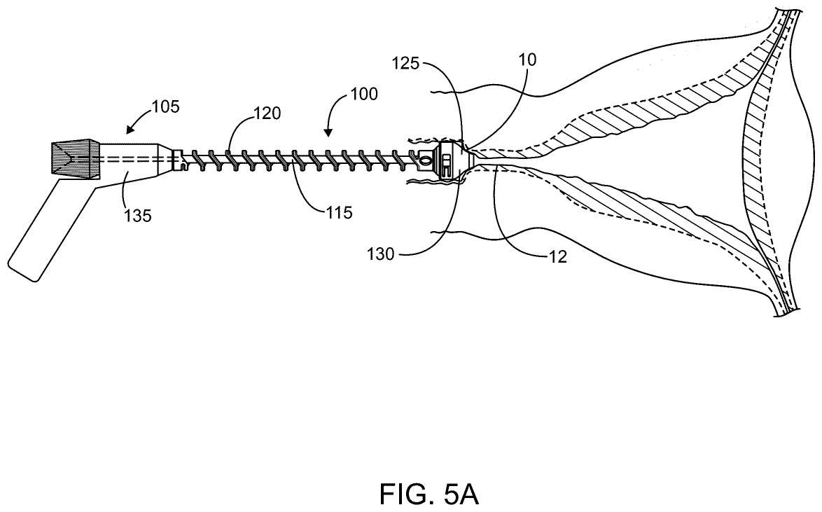

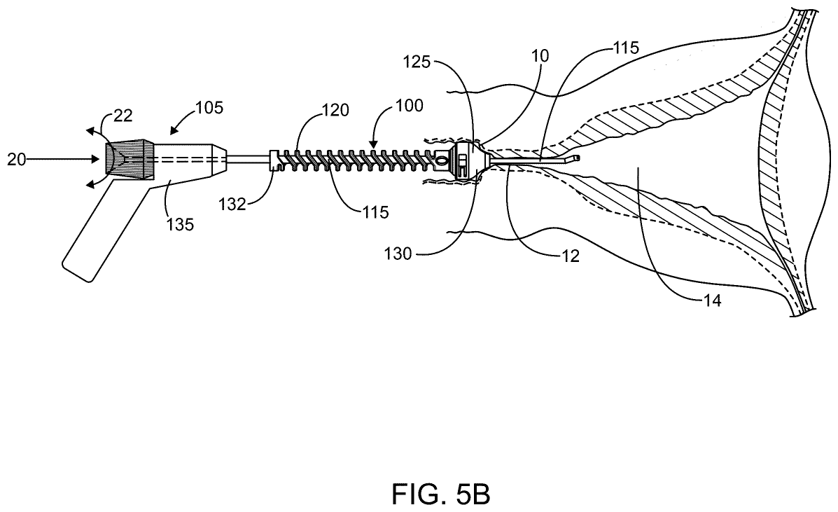

FIGS. 5A and 5B illustrate an example of using the cervical seal 100 with an endoscope 105. As noted herein, the cervical seal 100 can be used in any tissue cavity with an opening where an endoscopic device is used and a fluid seal is required. The variations shown in FIGS. 5A and 5B, where the device 100 is used against a cervix 10 is intended for illustrative purposes.

FIG. 5A shows an endoscope 105 having a cervical seal device 100 positioned about a shaft 115 of the endoscope 105. As shown, the spring structure 120 of the cervical seal device 100 is located about the shaft 115 with a proximal portion of the spring structure 120 engaging the endoscope 105 (in this variation, the device 100 engages a handle 135 of the endoscope 105). The sealing member 125 of the device 100 is located at the distal end of the body of the device 100 and includes an external sealing surface 130 that engages a tissue surface, which in this example comprises a cervix 10).

FIGS. 5A and 5B illustrate an example of endoscopically examining a cavity (i.e., the uterus 14) within a body of a patient by advancing the endoscope 105 within the body. The endoscope 105 includes a sealing body 100 positioned over a shaft 115 of the endoscope 105 with a sealing member 125 of the sealing body 100 located at a distal portion of the shaft 115. A proximal end of the sealing body 100 extends proximally from the sealing member 125 and comprises a spring structure 120.

The endoscope 105 can be positioned the body such that the sealing member 125 forms a fluid seal against the tissue surface of the cervix 10 about an opening, which in this case is the cervical canal or cavity 12. FIG. 5B shows the advancement of the endoscope 105, causing the shaft 115 to advance within the sealing device 100. Typically, and as discussed above, a portion of the endoscope 105 (e.g., a handle 135) compresses the helical spring structure 120 to apply a force of the sealing member 125 against the tissue 10. FIG. 5B also shows the helical spring structure 120 being axially compressed in direction 20, where a hub 132 of the spring structure 120 is locked against the shaft 115 to increase a compressive force on the helical spring structure 120 to increase a force of the sealing surface 130 against tissue 10 to maintain the fluid seal of the sealing member against the tissue surface.

As noted above, the sealing member can include a configuration that allows rotation of the shaft 115/endoscope 105, as shown by rotational direction 22, without rotating a surface 130 of the sealing member 125. This permits rotation of the distal end of the endoscope shaft 115.

Each of the individual variations or embodiments described and illustrated herein has discrete components and features that may be readily separated from or combined with the features of any of the other variations or embodiments. Modifications may be made to adapt a particular situation, material, composition of matter, process, process act(s), or step(s) to the objective(s), spirit, or scope of the present invention.

All existing subject matter mentioned herein (e.g., publications, patents, and patent applications) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

This disclosure is not intended to be limited to the scope of the particular forms set forth but is intended to cover alternatives, modifications, and equivalents of the variations or embodiments described herein. Further, the scope of the disclosure fully encompasses other variations or embodiments that may become obvious to those skilled in the art in view of this disclosure.

What is claimed is:

1. A cervical seal for use with an endoscope during transcervical access procedures, comprising:
an elongate body adapted to fit over a shaft of an endoscope;
a sealing member disposed at a distal end of the elongate body, the sealing member having a distal surface with a tapered configuration that tapers in a distal direction; and
the elongate body having a helical spring configuration such that the elongate body can compress when a force is applied and expand upon reduction of the force, a proximal end of the elongate body adapted to abut against a handle of the endoscope and a distal end of the helical spring configuration connected to the sealing member, wherein the elongate body is configured to bias the sealing member toward a cervix of a patient to maintain sealing engagement with the cervix.

2. The cervical seal of claim 1, wherein the sealing member comprises a housing carrying an elastomeric seal configured to create a seal against the shaft extending through an aperture in the sealing member.

3. The cervical seal of claim 1, wherein the sealing member comprises an internal housing located within an external housing, where the external housing comprises the tapered configuration and is configured to maintain a sealing engagement with the cervix.

4. The cervical seal of claim 3, wherein the internal housing and external housing are configured to rotate relative to each other about a longitudinal axis such that when engaged against the cervix, the external housing remains stationary upon rotation of the internal housing.

5. The cervical seal of claim 1, further comprising a collar located at the proximal end of the elongate body, the collar configured to abut a portion of a handle of the endoscope.

6. The cervical seal of claim 5, wherein the collar includes a locking mechanism having a locking surface that can be removably locked against the shaft.

7. The cervical seal of claim 6, wherein the locking mechanism permits locking the collar on the shaft after shortening of an axial distance between the proximal end of the elongate body and the sealing member.

8. The cervical seal of claim 6, wherein the locking mechanism comprises a rocker structure, where the locking surface is positioned within the collar.

9. The cervical seal of claim 1, wherein the distal surface comprises a soft material to improve conformability of the distal surface against the cervix.

10. A method of endoscopically examining a cavity within a body of an patient, the method comprising:
advancing an endoscope within the body, the endoscope having a sealing body positioned over a shaft of the endoscope with a sealing member of the sealing body located at a distal portion of the shaft and a proximal end of the sealing body extending proximally from the sealing member and comprising a spring structure;
positioning the endoscope within the body such that the sealing member forms a fluid seal against a tissue surface about an opening within the cavity; and
advancing the shaft through the sealing member such that the spring structure compresses while the shaft is moved therethrough and causes the sealing member to apply force against the tissue surface to maintain the fluid seal of the sealing member against the tissue surface.

11. The method of claim 10, further comprising compressing the spring structure in a longitudinal direction and locking a proximal end of the sealing body to a portion of the shaft to increase force applied by the sealing member against the tissue surface.

12. The method of claim 10, wherein the sealing member comprises an outer body and an inner body, and moving the shaft axially within the inner body against a shaft seal within the inner body such that the shaft seal maintains a fluid seal against the shaft.

13. The method of claim 12, further comprising rotating the shaft and inner body relative to the outer body such that the outer body remains stationary against the tissue surface.
